(19) United States
(12) Patent Application Publication
(10) Pub. No.: US 2022/0139504 A1
(43) Pub. Date: May 5, 2022

(54) Systems and Methods for Predicting the Olfactory Properties of Molecules Using Machine Learning

(71) Applicant: Google LLC, Mountain View, CA (US)

(72) Inventors: Alexander Wiltschko, Somerville, MA (US); Benjamin Manuel Sanchez-Lengeling, Somerville, MA (US)

(21) Appl. No.: 17/429,192

(22) PCT Filed: Feb. 10, 2020
(86) PCT No.: PCT/US2020/017477

§ 371 Date: Aug. 6, 2021

Related U.S. Application Data
(60) Provisional application No. 62/803,092, filed on Feb. 8, 2019.

(51) Int. Cl. G16C 20/30; G06N 3/08; G16C 20/70; G16C 20/80

(52) U.S. Cl. CPC G16C 20/30; G16C 20/80; G16C 20/70; G06N 3/08

(57) ABSTRACT

The present disclosure provides systems and methods for predicting olfactory properties of a molecule. One example method includes obtaining a machine-learned graph neural network trained to predict olfactory properties of molecules based at least in part on chemical structure data associated with the molecules. The method includes obtaining a graph that graphically describes a chemical structure of a selected molecule. The method includes providing the graph as input to the machine-learned graph neural network. The method includes receiving prediction data descriptive of one or more predicted olfactory properties of the selected molecule as an output of the machine-learned graph neural network. The method includes providing the prediction data descriptive of the one or more predicted olfactory properties of the selected molecule as an output.

### FIELD

The present disclosure relates generally to machine learning. More particularly, the present disclosure relates to the use of machine-learned models to predict olfactory properties of molecules.

### BACKGROUND

The relationship between a molecule's structure and its olfactory perceptual properties (e.g., the scent of a molecule as observed by a human) is complex, and, to date, generally little is known about such relationships. For example, the flavor and fragrance industries generally rely on trial-and-error, heuristics, and/or mining natural products to provide commercially useful products having desired olfactory properties. There is generally a lack of meaningful principles for organizing the olfactory environment, though it is known that mapping between molecular structure and scent can be very nonlinear, such that small changes in molecules can yield large changes in olfactory quality. Additionally, the inverse can also be true, where diverse families of molecules all can smell the same.

### SUMMARY

Aspects and advantages of embodiments of the present disclosure will be set forth in part in the following description, or can be learned from the description, or can be learned through practice of the embodiments.

One example aspect of the present disclosure is directed to a computer-implemented method for predicting olfactory properties of molecules. The method includes obtaining, by one or more computing devices, a machine-learned graph neural network trained to predict olfactory properties of molecules based at least in part on chemical structure data associated with the molecules. The method includes obtaining, by the one or more computing devices, a graph that graphically describes a chemical structure of a selected molecule. The method includes providing, by the one or more computing devices, the graph that graphically describes the chemical structure of the selected molecule as input to the machine-learned graph neural network. The method includes receiving, by the one or more computing devices, prediction data descriptive of one or more predicted olfactory properties of the selected molecule as an output of the machine-learned graph neural network. The method includes providing, by the one or more computing devices, the prediction data descriptive of the one or more predicted olfactory properties of the selected molecule as an output.

Another example aspect of the present disclosure is directed to a computing device. The computing device includes one or more processors; and one or more non-transitory computer-readable media that store instructions. The instructions, when executed by the one or more processors, cause the computing device to perform operations. The operations include obtaining a machine-learned graph neural network trained to predict one or more olfactory properties of a molecule based at least in part on chemical structure data associated with the molecule. The operations include obtaining graph data representative of a chemical structure of a selected molecule. The operations include providing the graph data representative of the chemical structure as input to the machine-learned graph neural network. The operations include receiving prediction data descriptive of one or more olfactory properties associated with the selected molecule as an output of the machine-learned graph neural network. The operations include providing the prediction data descriptive of the one or more predicted olfactory properties of the selected molecule as an output.

Other aspects of the present disclosure are directed to various systems, apparatuses, non-transitory computer-readable media, user interfaces, and electronic devices.

These and other features, aspects, and advantages of various embodiments of the present disclosure will become better understood with reference to the following description and appended claims. The accompanying drawings, which are incorporated in and constitute a part of this specification, illustrate example embodiments of the present disclosure and, together with the description, serve to explain the related principles.

### BRIEF DESCRIPTION OF THE DRAWINGS

Detailed discussion of embodiments directed to one of ordinary skill in the art is set forth in the specification, which makes reference to the appended figures, in which:

FIG. 1A depicts a block diagram of an example computing system according to example embodiments of the present disclosure;

FIG. 1B depicts a block diagram of an example computing device according to example embodiments of the present disclosure;

FIG. 1C depicts a block diagram of an example computing device according to example embodiments of the present disclosure;

FIG. 2 depicts a block diagram of an example prediction model according to example embodiments of the present disclosure;

FIG. 3 depicts a block diagram of an example prediction model according to example embodiments of the present disclosure;

FIG. 4 depicts a flowchart diagram of example operations for prediction of molecule olfactory properties according to example embodiments of the present disclosure; and

FIG. 5 depicts example illustrations for visualizing structural contribution associated with predicted olfactory properties according to example embodiments of the present disclosure.

FIG. 6 illustrates an example model schematic and data flow according to example embodiments of the present disclosure.

FIG. 7 illustrates the global structure of an example learned embedding space according to example embodiments of the present disclosure.

Reference numerals that are repeated across plural figures are intended to identify the same features in various implementations.

### DETAILED DESCRIPTION

### Overview

Example aspects of the present disclosure are directed to systems and methods that include or otherwise leverage machine-learned models (e.g., graph neural networks) in conjunction with molecule chemical structure data to predict one or more perceptual (e.g., olfactory, gustatory, tactile, etc.) properties of a molecule. In particular, the systems and methods of the present disclosure can predict the olfactory properties (e.g., humanly-perceived odor expressed using labels such as “sweet,” “piney,” “pear,” “rotten,” etc.) of a single molecule based on the chemical structure of the molecule. According to an aspect of the present disclosure, in some implementations, a machine-learned graph neural network can be trained and used to process a graph that graphically describes the chemical structure of a molecule to predict olfactory properties of the molecule. In particular, the graph neural network can operate directly upon the graph representation of the chemical structure of the molecule (e.g., perform convolutions within the graph space) to predict the olfactory properties of the molecule. As one example, the graph can include nodes that correspond to atoms and edges that correspond to chemical bonds between the atoms. Thus, the systems and methods of the present disclosure can provide prediction data that predicts the smell of previously unassessed molecules through the use of machine-learned models. The machine-learned models can be trained, for example, using training data that includes descriptions of molecules (e.g., structural descriptions of molecules, graph-based descriptions of chemical structures of molecules, etc.) that have been labeled (e.g., manually by an expert) with descriptions of olfactory properties (e.g., textual descriptions of odor categories such as “sweet,” “piney,” “pear,” “rotten,” etc.) that have been assessed for the molecules.

Thus, aspects of the present disclosure are directed to propose the use of graph neural networks for quantitative structure-odor relationship (QSOR) modeling. Example implementations of the systems and methods described herein significantly outperform prior methods on a novel data set labeled by olfactory experts. Additional analysis shows that the learned embeddings from graph neural networks capture a meaningful odor space representation of the underlying relationship between structure and odor.

More particularly, the relationship between a molecule's structure and its olfactory perceptual properties (e.g., the scent of a molecule as observed by a human) is complex, and, to date, generally little is known about such relationships. Accordingly, the systems and methods of the present disclosure provide for the use of deep learning and under-utilized data sources to obtain predictions of olfactory perceptual properties of unseen molecules, thus allowing for improvements in the identification and development of molecules having desired perceptual properties, for example, allowing for development of new compounds useful in commercial flavor, fragrance, or cosmetics products, improving expertise in prediction of drug psychoactive effects from single molecules, and/or the like. The improved systems for predicting of olfactory perceptual properties of molecules described herein can provide significant improvements in the identification and development of molecules having desired perceptual properties and the development of new useful compounds.

More particularly, according to one aspect of the present disclosure, machine-learned models, such as graph neural network models, can be trained to provide predictions of perceptual properties (e.g., olfactory properties, gustatory properties, tactile properties, etc.) of a molecule based on an input graph of the chemical structure of the molecule. For instance, a machine-learned model may be provided with an input graph structure of a molecule's chemical structure, for example, based on a standardized description of a molecule's chemical structure (e.g., a simplified molecular-input line-entry system (SMILES) string, etc.). The machine-learned model may provide output comprising a description of predicted perceptual properties of the molecule, such as, for example, a list of olfactory perceptual properties descriptive of what the molecule would smell like to a human. For instance, a SMILES string can be provided, such as the SMILES string “O═C(OCCC(C)C)C” for the chemical structure of isoamyl acetate, and the machine-learned model can provide as output a description of what that molecule would smell like to a human, for example, a description of the molecule's odor properties such as “fruit, banana, apple”. In particular, in some embodiments, in response to receipt of a SMILES string or other description of chemical structure, the systems and methods of the present disclosure can convert the string to a graph structure that graphically describes the two-dimensional structure of a molecule and can provide the graph structure to a machine-learned model (e.g., a trained graph convolutional neural network and/or other type of machine-learned model) that can predict, from either the graph structure or features derived from the graph structure, olfactory properties of the molecule. Additionally or alternatively to the two-dimensional graph, systems and methods could provide for creating a three-dimensional graph representation of the molecule, for example using quantum chemical calculations, for input to a machine-learned model.

In some examples, the prediction can indicate whether or not the molecule has a particular desired olfactory perceptual quality (e.g., a target scent perception, etc.). In some embodiments, the prediction data can include one or more types of information associated with a predicted olfactory property of a molecule. For instance, prediction data for a molecule can provide for classifying the molecule into one olfactory property class and/or into multiple olfactory property classes. In some instances, the classes can include human-provided (e.g., experts) textual labels (e.g., sour, cherry, piney, etc.). In some instances, the classes can include non-textual representations of scent/odor, such as a location on a scent continuum or the like. In some instances, prediction data for molecules can include intensity values that describe the intensity of the predicted scent/odor. In some instances, prediction data can include confidence values associated with the predicted olfactory perceptual property.

In addition or alternatively to specific classifications for a molecule, prediction data can include a numerical embedding that allows for similarity search, clustering, or other comparisons between two or more molecules based on a measure of distance between two or more embeddings. For example, in some implementations, the machine-learned model can be trained to output embeddings that can be used to measure similarity by training the machine-learned model using a triplet training scheme where the model is trained to output embeddings that are closer in the embedding space for a pair of similar chemical structures (e.g., an anchor example and a positive example) and to output embeddings that are more distant in the embedding space for a pair of dissimilar chemical structures (e.g., the anchor and a negative example).

Thus, in some implementations, the systems and methods of the present disclosure may not necessitate the generation of feature vectors descriptive of the molecule for input to a machine-learned model. Rather, the machine-learned model can be provided directly with the input of a graph-value form of the original chemical structure, thus reducing the resources required to make olfactory property predictions. For example, by providing for the use of the graph structure of molecules as input to the machine-learned model, new molecule structures can be conceptualized and evaluated without requiring the experimental production of such molecule structures to determine perceptual properties, thereby greatly accelerating the ability to evaluate new molecular structure and saving significant resources.

According to another aspect of the present disclosure, training data comprising a plurality of known molecules can be obtained to provide for training one or more machine-learned models (e.g., a graph convolutional neural network, other type of machine-learned model) to provide predictions of olfactory properties of molecules. For example, in some embodiments, the machine-learned models can be trained using one or more datasets of molecules, where the dataset includes the chemical structure and a textual description of the perceptual properties (e.g., descriptions of the smell of the molecule provided by human experts, etc.) for each molecule. As one example, the training data can be derived from industry lists such as, for example, perfume industry lists of chemical structures and their corresponding odors. In some embodiments, due to the fact that some perceptual properties are rare, steps can be taken to balance out common perceptual properties and rare perceptual properties when training the machine-learned model(s).

According to another aspect of the present disclosure, in some embodiments, the systems and methods may provide for indications of how changes to a molecule structure could affect the predicted perceptual properties. For example, the systems and methods could provide indications of how changes to the molecule structure may affect the intensity of a particular perceptual property, how catastrophic a change in the molecule's structure would be to desired perceptual qualities, and/or the like. In some embodiments, the systems and methods may provide for adding and/or removing one or more atoms and/or groups of atoms from a molecule's structure to determine the effect of such addition/removal on one or more desired perceptual properties. For example, iterative and different changes to the chemical structure can be performed and then the result can be evaluated to understand how such change would affect the perceptual properties of the molecule. As yet another example, a gradient of the classification function of the machine-learned model can be evaluated (e.g., with respect to a particular label) at each node and/or edge of the input graph (e.g., via backpropagation through the machine-learned model) to generate a sensitivity map (e.g., that indicates how important each node and/or edge of the input graph was for output of such particular label). Further, in some implementations, a graph of interest can be obtained, similar graphs can be sampled by adding noise to the graph, and then the average of the resulting sensitivity maps for each sampled graph can be taken as the sensitivity map for the graph of interest. Similar techniques can be performed to determine perceptual differences between different molecule structures.

According to another aspect, the systems and methods of the present disclosure can provide for interpreting and/or visualizing which aspects of a molecule's structure most contributes to its predicted odor quality. For example, in some embodiments, a heat map could be generated to overlay the molecule structure that provides indications of which portions of a molecule's structure are most important to the perceptual properties of the molecule and/or which portions of a molecule's structure are less important to the perceptual properties of the molecule. In some implementations, data indicative of how changes to a molecule structure would impact olfactory perception can be used to generate visualizations of how the structure contributes to a predicted olfactory quality. For example, as described above, iterative changes to the molecule's structure (e.g., a knock-down technique, etc.) and their corresponding outcomes can be used to evaluate which portions of the chemical structure are most contributory to the olfactory perception. As another example, as described above, a gradient technique can be used to generate a sensitivity map for the chemical structure, which can then be used to produce the visualization (e.g., in the form of a heat map).

According to another aspect of the present disclosure, in some embodiments, machine-learned model(s) may be trained to produce predictions of a molecule chemical structure that would provide one or more desired perceptual properties (e.g., generate a molecule chemical structure that would produce a particular scent quality, etc.). For example, in some implementations, an iterative search can be performed to identify proposed molecule(s) that are predicted to exhibit one or more desired perceptual properties (e.g., targeted scent quality, intensity, etc.). For instance, an iterative search can propose a number of candidate molecule chemical structures that can be evaluated by the machine-learned model(s). In one example, candidate molecule structures can be generated through an evolutionary or genetic process. As another example, candidate molecule structures can be generated by a reinforcement learning agent (e.g., recurrent neural network) that seeks learn a policy that maximizes a reward that is a function of whether the generated candidate molecule structures exhibit the one or more desired perceptual properties.

Thus, in some implementations, a plurality of candidate molecule graph structures that describe the chemical structure of each candidate molecule can be generated (e.g., iteratively generated) for use as input to a machine-learned model. The graph structure for each candidate molecule can be input to the machine-learned model to be evaluated. The machine-learned model can produce prediction data for each candidate molecule that describes one or more perceptual properties of the candidate molecule. The candidate molecule prediction data can then be compared to the one or more desired perceptual properties to determine if the candidate molecule would exhibit desired perceptual properties (e.g., a viable molecule candidate, etc.). For example, the comparison can be performed to generate a reward (e.g., in a reinforcement learning scheme) or to determine whether to retain or discard the candidate molecule (e.g., in an evolutionary learning scheme). Brute force search approaches may also be employed. In further implementations, which may or may not have the evolutionary or reinforcement learning structures described above, the search for candidate molecules that exhibit the one or more desired perceptual properties can be structured as a multi-parameter optimization problem with a constraint on the optimization defined for each desired property.

According to another aspect of the present disclosure, systems and methods may provide for predicting, identifying, and/or optimizing other properties associated with a molecule structure along with desired olfactory properties. For example, the machine-learned model(s) may predict or identify properties of molecule structures such as optical properties (e.g., clarity, reflectiveness, color, etc.), gustatory properties (e.g., tastes like “banana,” “sour,” “spicy,” etc.) shelf-stability, stability at particular pH levels, biodegradability, toxicity, industrial applicability, and/or the like.

According to another aspect of the present disclosure, the machine-learned models described herein can be used in active learning techniques to narrow a wide field of candidates to a smaller set of molecules that are then manually evaluated. According to other aspects of the present disclosure, systems and methods can allow for synthesis of molecules with particular properties in an iterative design-test-refine process. For example, based on prediction data from the machine-learned models, molecules can be proposed for development. The molecules can then be synthesized, and then can be subjected to specialized testing. Feedback from the testing can then be provided back to the design phase to refine the molecules to better achieve desired properties, etc.

The systems and methods of the present disclosure provide a number of technical effects and benefits. As one example, the systems and methods described herein can allow for reducing the time and resources required to determine whether a molecule would provide desired perceptual qualities. For instance, the systems and methods described herein allow for using graph structures descriptive of the chemical structure of a molecule rather than necessitating the generation of feature vectors describing a molecule to provide for model input. Thus, the systems and methods provide technical improvements in the resources required to obtain and analyze model inputs and produce model prediction outputs. Furthermore, the use of machine-learned models to predict olfactory properties represents the integration of machine learning into a practical application (e.g., predicting olfactory properties). That is, the machine-learned models are adapted to the specific technical implementation of predicting olfactory properties.

With reference now to the Figures, example embodiments of the present disclosure will be discussed in further detail.

### Example Devices and Systems

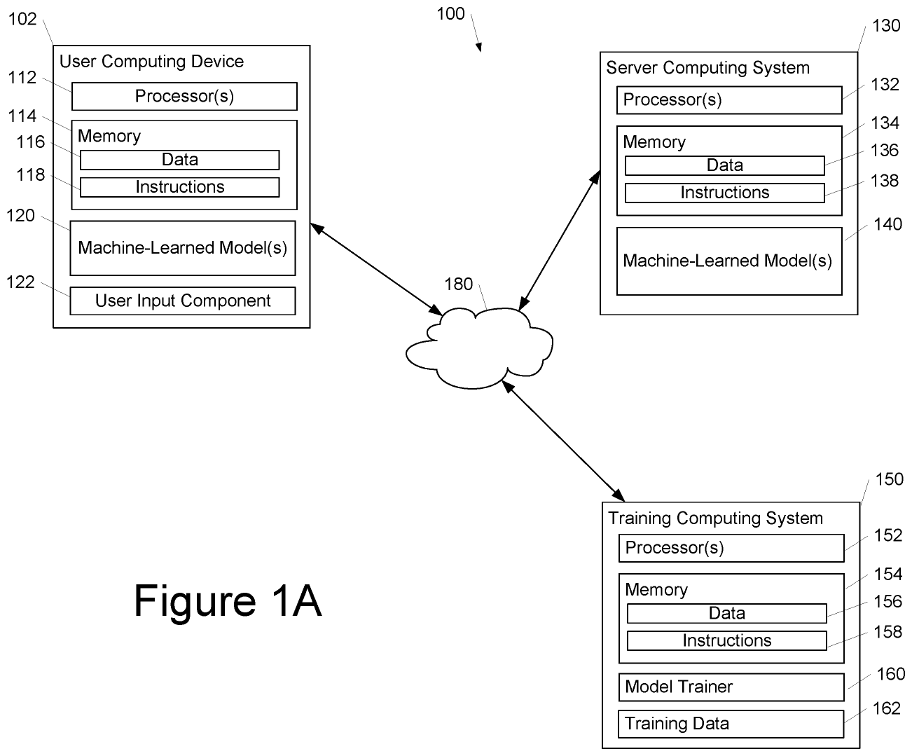

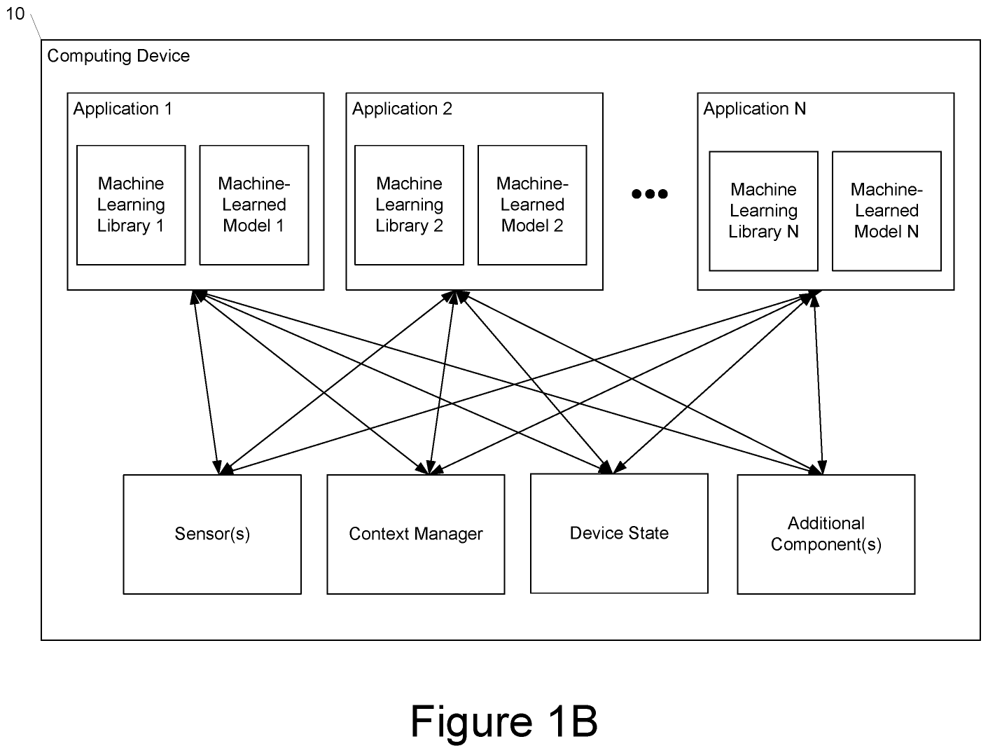

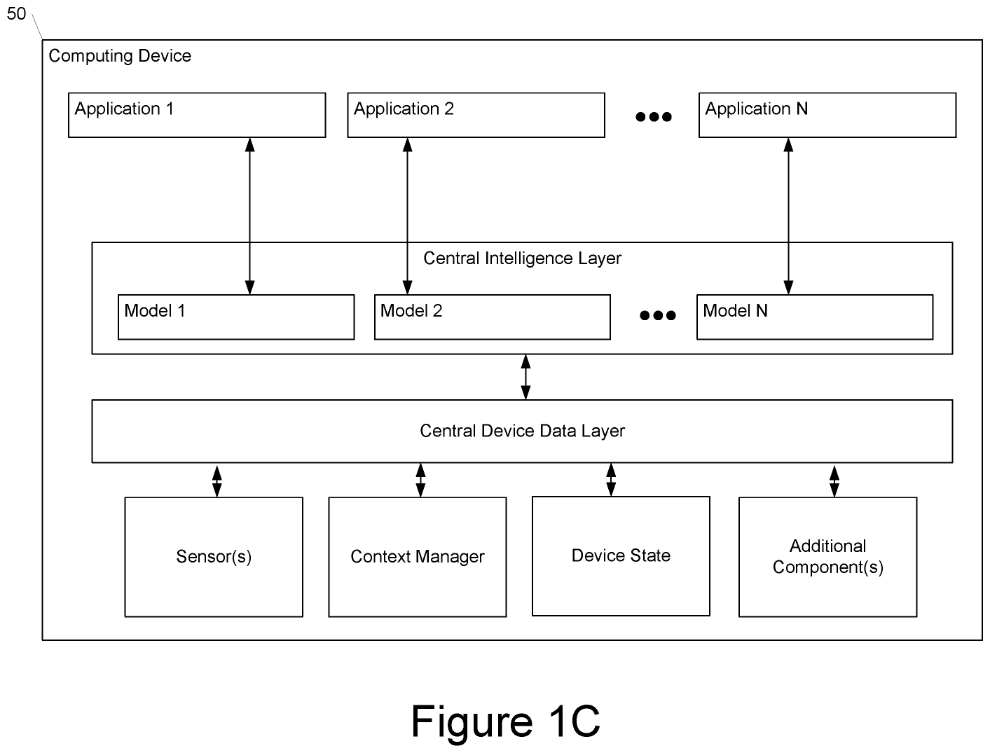

FIG. 1A depicts a block diagram of an example computing system 100 that can facilitate predictions of perceptual properties, such as olfactory perceptual properties, of molecules according to example embodiments of the present disclosure. The system 100 is provided as one example only. Other computing systems that include different components can be used in addition or alternatively to the system 100. The system 100 includes a user computing device 102, a server computing system 130, and a training computing system 150 that are communicatively coupled over a network 180.

The user computing device 102 can be any type of computing device, such as, for example, a personal computing device (e.g., laptop or desktop), a mobile computing device (e.g., smartphone or tablet), a gaming console or controller, a wearable computing device, an embedded computing device, or any other type of computing device.

The user computing device 102 includes one or more processors 112 and a memory 114. The one or more processors 112 can be any suitable processing device (e.g., a processor core, a microprocessor, an ASIC, a FPGA, a controller, a microcontroller, etc.) and can be one processor or a plurality of processors that are operatively connected. The memory 114 can include one or more non-transitory computer-readable storage mediums, such as RAM, ROM, EEPROM, EPROM, flash memory devices, magnetic disks, etc., and combinations thereof. The memory 114 can store data 116 and instructions 118 which are executed by the processor 112 to cause the user computing device 102 to perform operations.

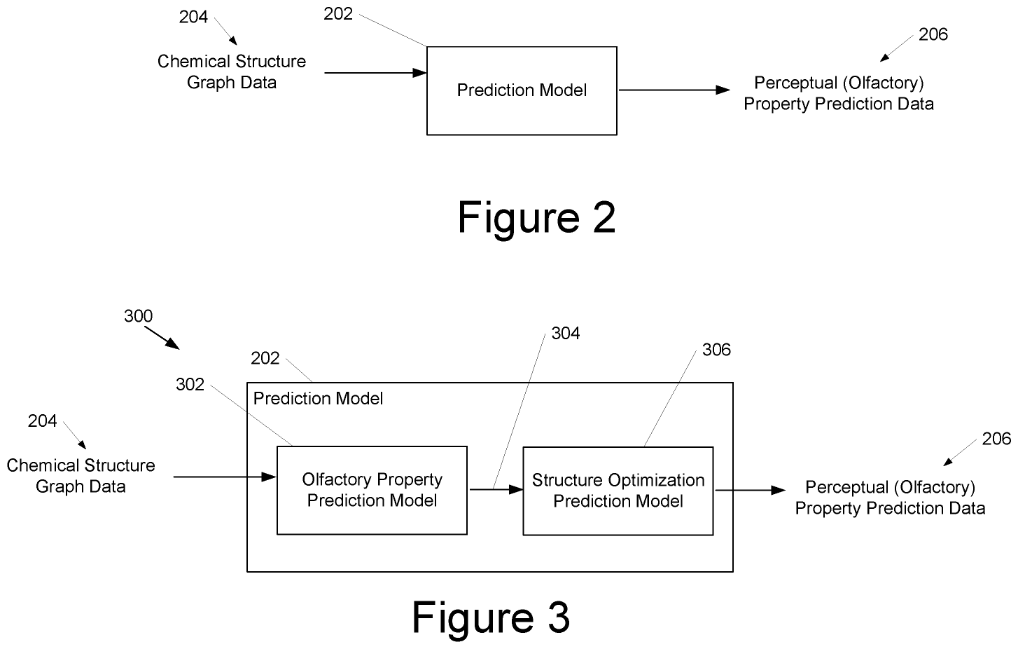

In some implementations, the user computing device 102 can store or include one or more machine-learned models 120, such as an olfactory property prediction machine-learned model as discussed herein. For example, the machine-learned models 120 can be or can otherwise include various machine-learned models such as neural networks (e.g., deep neural networks) or other types of machine-learned models, including non-linear models and/or linear models. Neural networks can include feed-forward neural networks, recurrent neural networks (e.g., long short-term memory recurrent neural networks), convolutional neural networks or other forms of neural networks. Example machine-learned models 120 are discussed with reference to FIGS. 2 and 3.

In some implementations, the one or more machine-learned models 120 can be received from the server computing system 130 over network 180, stored in the user computing device memory 114, and then used or otherwise implemented by the one or more processors 112. In some implementations, the user computing device 102 can implement multiple parallel instances of a single machine-learned model 120.

Additionally or alternatively, one or more machine-learned models 140 can be included in or otherwise stored and implemented by the server computing system 130 that communicates with the user computing device 102 according to a client-server relationship. For example, the machine-learned models 140 can be implemented by the server computing system 140 as a portion of a web service. Thus, one or more models 120 can be stored and implemented at the user computing device 102 and/or one or more models 140 can be stored and implemented at the server computing system 130.

The user computing device 102 can also include one or more user input component 122 that receives user input. For example, the user input component 122 can be a touch-sensitive component (e.g., a touch-sensitive display screen or a touch pad) that is sensitive to the touch of a user input object (e.g., a finger or a stylus). The touch-sensitive component can serve to implement a virtual keyboard. Other example user input components include a microphone, a traditional keyboard, a camera, or other means by which a user can provide user input.

The server computing system 130 includes one or more processors 132 and a memory 134. The one or more processors 132 can be any suitable processing device (e.g., a processor core, a microprocessor, an ASIC, a FPGA, a controller, a microcontroller, etc.) and can be one processor or a plurality of processors that are operatively connected. The memory 134 can include one or more non-transitory computer-readable storage mediums, such as RAM, ROM, EEPROM, EPROM, flash memory devices, magnetic disks, etc., and combinations thereof. The memory 134 can store data 136 and instructions 138 which are executed by the processor 132 to cause the server computing system 130 to perform operations.

In some implementations, the server computing system 130 includes or is otherwise implemented by one or more server computing devices. In instances in which the server computing system 130 includes plural server computing devices, such server computing devices can operate according to sequential computing architectures, parallel computing architectures, or some combination thereof.

As described above, the server computing system 130 can store or otherwise include one or more machine-learned models 140. For example, the models 140 can be or can otherwise include various machine-learned models, such as olfactory property prediction machine-learned models. Example machine-learned models include neural networks or other multi-layer non-linear models. Example neural networks include feed forward neural networks, deep neural networks, recurrent neural networks, and convolutional neural networks. Example models 140 are discussed with reference to FIGS. 2 through 4.

The user computing device 102 and/or the server computing system 130 can train the models 120 and/or 140 via interaction with the training computing system 150 that is communicatively coupled over the network 180. The training computing system 150 can be separate from the server computing system 130 or can be a portion of the server computing system 130.

The training computing system 150 includes one or more processors 152 and a memory 154. The one or more processors 152 can be any suitable processing device (e.g., a processor core, a microprocessor, an ASIC, a FPGA, a controller, a microcontroller, etc.) and can be one processor or a plurality of processors that are operatively connected. The memory 154 can include one or more non-transitory computer-readable storage mediums, such as RAM, ROM, EEPROM, EPROM, flash memory devices, magnetic disks, etc., and combinations thereof. The memory 154 can store data 156 and instructions 158 which are executed by the processor 152 to cause the training computing system 150 to perform operations. In some implementations, the training computing system 150 includes or is otherwise implemented by one or more server computing devices.

The training computing system 150 can include a model trainer 160 that trains the machine-learned models 120 and/or 140 stored at the user computing device 102 and/or the server computing system 130 using various training or learning techniques, such as, for example, backwards propagation of errors. In some implementations, performing backwards propagation of errors can include performing truncated backpropagation through time. The model trainer 160 can perform a number of generalization techniques (e.g., weight decays, dropouts, etc.) to improve the generalization capability of the models being trained.

In particular, the model trainer 160 can train the machine-learned models 120 and/or 140 based on a set of training data 162. The training data 162 can include, for example, descriptions of molecules (e.g., graphical descriptions of chemical structures of molecules) that have been labeled (e.g., manually by an expert) with descriptions of olfactory properties (e.g., textual descriptions of odor categories such as “sweet,” “piney,” “pear,” “rotten,” etc.) that have been assessed for the molecules, and/or the like.

The model trainer 160 includes computer logic utilized to provide desired functionality. The model trainer 160 can be implemented in hardware, firmware, and/or software controlling a general purpose processor. For example, in some implementations, the model trainer 160 includes program files stored on a storage device, loaded into a memory, and executed by one or more processors. In other implementations, the model trainer 160 includes one or more sets of computer-executable instructions that are stored in a tangible computer-readable storage medium such as RAM hard disk or optical or magnetic media.

The network 180 can be any type of communications network, such as a local area network (e.g., intranet), wide area network (e.g., Internet), or some combination thereof and can include any number of wired or wireless links. In general, communication over the network 180 can be carried via any type of wired and/or wireless connection, using a wide variety of communication protocols (e.g., TCP/IP, HTTP, SMTP, FTP), encodings or formats (e.g., HTML, XML), and/or protection schemes (e.g., VPN, secure HTTP, SSL).

FIG. 1A illustrates one example computing system that can be used to implement the present disclosure. Other computing systems can be used as well. For example, in some implementations, the user computing device 102 can include the model trainer 160 and the training dataset 162. In such implementations, the models 120 can be both trained and used locally at the user computing device 102. Any components illustrated as being included in one of device 102, system 130, and/or system 150 can instead be included at one or both of the others of device 102, system 130, and/or system 150.

FIG. 1B depicts a block diagram of an example computing device 10 according to example embodiments of the present disclosure. The computing device 10 can be a user computing device or a server computing device.

The computing device 10 includes a number of applications (e.g., applications 1 through N). Each application contains its own machine learning library and machine-learned model(s). For example, each application can include a machine-learned model. Example applications include a text messaging application, an email application, a dictation application, a virtual keyboard application, a browser application, etc.

As illustrated in FIG. 1B, each application can communicate with a number of other components of the computing device, such as, for example, one or more sensors, a context manager, a device state component, and/or additional components. In some implementations, each application can communicate with each device component using an API (e.g., a public API). In some implementations, the API used by each application is specific to that application.

FIG. 1C depicts a block diagram of an example computing device 50 according to example embodiments of the present disclosure. The computing device 50 can be a user computing device or a server computing device.

The computing device 50 includes a number of applications (e.g., applications 1 through N). Each application is in communication with a central intelligence layer. Example applications include a text messaging application, an email application, a dictation application, a virtual keyboard application, a browser application, etc. In some implementations, each application can communicate with the central intelligence layer (and model(s) stored therein) using an API (e.g., a common API across all applications).

The central intelligence layer includes a number of machine-learned models. For example, as illustrated in FIG. 1C, a respective machine-learned model (e.g., a model) can be provided for each application and managed by the central intelligence layer. In other implementations, two or more applications can share a single machine-learned model. For example, in some implementations, the central intelligence layer can provide a single model (e.g., a single model) for all of the applications. In some implementations, the central intelligence layer is included within or otherwise implemented by an operating system of the computing device 50.

The central intelligence layer can communicate with a central device data layer. The central device data layer can be a centralized repository of data for the computing device 50. As illustrated in FIG. 1C, the central device data layer can communicate with a number of other components of the computing device, such as, for example, one or more sensors, a context manager, a device state component, and/or additional components. In some implementations, the central device data layer can communicate with each device component using an API (e.g., a private API).

### Example Model Arrangements

FIG. 2 depicts a block diagram of an example prediction model 202 according to example embodiments of the present disclosure. In some implementations, the prediction model 202 is trained to receive a set of input data 204 (e.g., molecule chemical structure graph data, etc.) and, as a result of receipt of the input data 204, provide output data 206, for example, olfactory property prediction data for the molecule.

FIG. 3 depicts a block diagram of an example machine-learned model 202 according to example embodiments of the present disclosure. The machine-learned model 202 is similar to prediction model 202 of FIG. 2 except that machine-learned model 202 of FIG. 3 is one example model that includes a olfactory property prediction model 302 and a molecule structure optimization prediction model 306. In some implementations, the machine-learned prediction model 202 can include a olfactory property prediction model 302 that predicts one or more olfactory perceptual properties for a molecule based on the chemical structure of the molecule (e.g., provided in a graph structure form) and a molecule structure optimization prediction model 306 that predicts how changes to a molecule structure could affect the predicted perceptual properties. Thus, the models might provide output that includes both olfactory perceptual properties and how a molecule structure affects those predicted olfactory properties.

### Example Methods

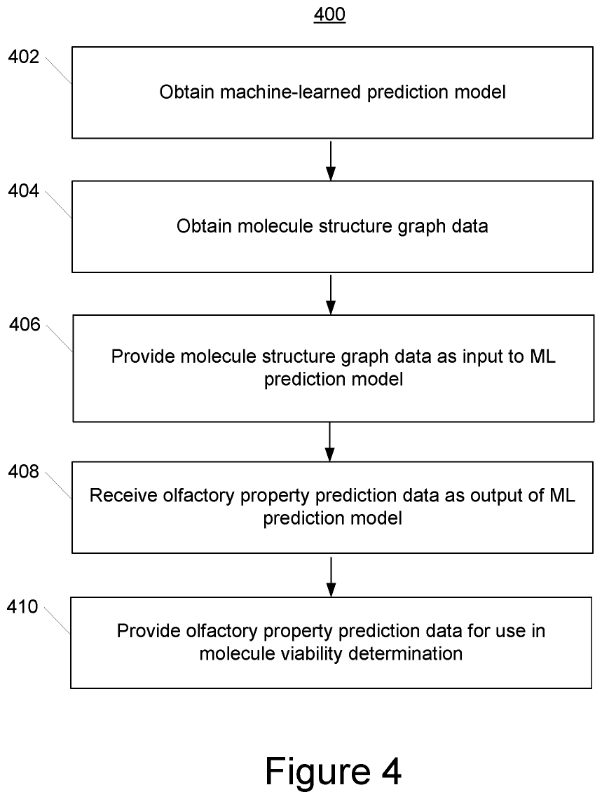

FIG. 4 depicts a flowchart diagram of example method 400 for predicting olfactory properties according to example embodiments of the present disclosure. Although FIG. 4 depicts steps performed in a particular order for purposes of illustration and discussion, the methods of the present disclosure are not limited to the particularly illustrated order or arrangement. The various steps of the method 400 can be omitted, rearranged, combined, and/or adapted in various ways without deviating from the scope of the present disclosure. Method 400 can be implemented by one or more computing devices, such as one or more of the computing devices depicted in FIGS. 1A-1C.

At 402, method 400 can include obtaining, by one or more computing devices, a machine-learned graph neural network trained to predict olfactory properties of molecules based at least in part on chemical structure data associated with the molecules. In particular, a machine-learned prediction model (e.g., graph neural network, etc.) can be trained and used to process a graph that graphically describes the chemical structure of a molecule to predict olfactory properties of the molecule. For example, a trained graph neural network can operate directly upon the graph representation of the chemical structure of the molecule (e.g., perform convolutions within the graph space) to predict the olfactory properties of the molecule. The machine-learned model can be trained using training data that includes descriptions of molecules (e.g., graphical descriptions of chemical structures of molecules) that have been labeled (e.g., manually by an expert) with descriptions of olfactory properties (e.g., textual descriptions of odor categories such as “sweet,” “piney,” “pear,” “rotten,” etc.) that have been assessed for the molecules. The trained machine-learned prediction model can provide prediction data that predicts the smell of previously unassessed molecules.

More particularly, most machine learning models require regularly-shaped input (e.g. a grid of pixels, or a vector of numbers) as input. However, GNNs enable the use of irregularly-shaped inputs, such as graphs, to be used directly in machine learning applications. As such, according to an aspect of the present disclosure, by viewing atoms as nodes, and bonds as edges, a molecule can be interpreted as a graph. Example GNNs are learnable permutation-invariant transformations on nodes and edges, which produce fixed-length vectors that are further processed by a fully-connected neural network. GNNs can be considered learnable featurizers specialized to a task, in contrast with expert-crafted general features.

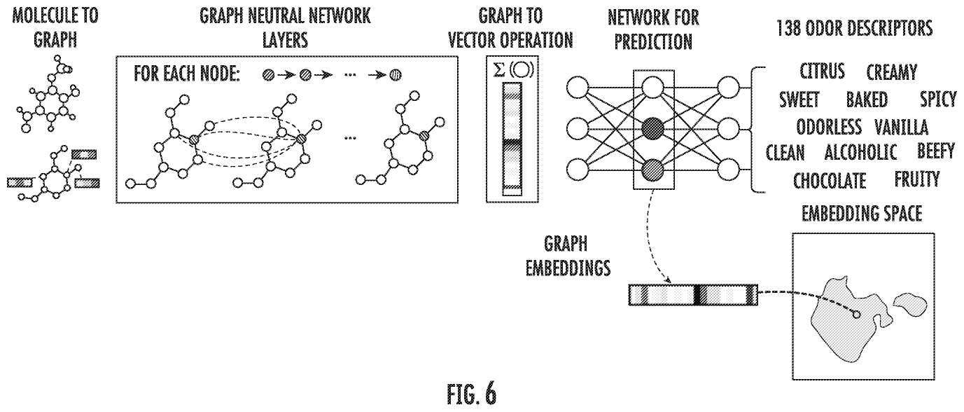

Some example GNNs include one or more message passing layers, each followed by a reduce-sum operation, followed by several fully connected layers. The example final fully-connected layer has a number of outputs equal to the number of odor descriptors being predicted. One example model is illustrated in FIG. 6, which illustrates an example model schematic and data flow. In the example illustrated in FIG. 6, each molecule is first featurized by its constituent atoms, bonds, and connectivities. Each Graph Neural Network (GNN) layer transforms the features from the previous layer. The outputs from the final GNN layer is reduced to a vector, which is then used for predicting odor descriptors via a fully-connected neural network. In some example implementations, graph embeddings can be retrieved from the penultimate layer of the model. An example of the embedding space representation for four odor descriptors is shown in the bottom right.

Referring again to FIG. 4, at 404, method 400 can include obtaining, by the one or more computing devices, a graph that graphically describes a chemical structure of a selected molecule. For instance, an input graph structure of a molecule's chemical structure (e.g., a previously unassessed molecule, etc.) can be obtained for use in predicting one or more perceptual (e.g., olfactory) properties of the molecule. For example, in some embodiments, a graph structure can be obtained based on a standardized description of a molecule's chemical structure, such as a simplified molecular-input line-entry system (SMILES) string, and/or the like. In some embodiments, in response to receipt of a SMILES string or other description of chemical structure, the one or more computing devices can convert the string to a graph structure that graphically describes the two-dimensional structure of a molecule. Additionally or alternatively, the one or more computing devices could provide for creating a three-dimensional representation of the molecule, for example using quantum chemical calculations, for input to a machine-learned model.

At 406, method 400 can include providing, by the one or more computing devices, the graph that graphically describes the chemical structure of the selected molecule as input to the machine-learned graph neural network. For example, the graph structure descriptive of a molecule's chemical structure, obtained at 404, can be provided to a machine-learned model (e.g., a trained graph convolutional neural network and/or other type of machine-learned model) that can predict, from either the graph structure or features derived from the graph structure, olfactory properties of the molecule.

At 408, method 400 can include receiving, by the one or more computing devices, prediction data descriptive of one or more predicted olfactory properties of the selected molecule as an output of the machine-learned graph neural network. In particular, the machine-learned model may provide output prediction data comprising a description of predicted perceptual properties of the molecule, such as, for example, a list of olfactory perceptual properties descriptive of what the molecule would smell like to a human. For instance, a SMILES string can be provided, such as the SMILES string “O=C(OCCC(C)C)C” for the chemical structure of isoamyl acetate, and the machine-learned model can provide as output a description of what that molecule would smell like to a human, for example, a description of the molecule's odor properties such as “fruit, banana, apple”.

In some example embodiments, the prediction data can indicate whether or not the molecule has a particular desired olfactory perceptual quality (e.g., a target scent perception, etc.). In some example embodiments, the prediction data can include one or more types of information associated with a predicted olfactory property of a molecule. For instance, prediction data for a molecule can provide for classifying the molecule into one olfactory property class and/or into multiple olfactory property classes. In some instances, the classes can include human-provided (e.g., experts) textual labels (e.g., sour, cherry, piney, etc.). In some instances, the classes can include non-textual representations of scent/odor, such as a location on a scent continuum or the like. In some example embodiments, prediction data for molecules can include intensity values that describe the intensity of the predicted scent/odor. In some example embodiments, prediction data can include confidence values associated with the predicted olfactory perceptual property. In some example embodiments, in addition or alternatively to specific classifications for a molecule, prediction data can include numerical embedding that allows for similar search or other comparisons between two molecules based on a measure of distance between two embeddings.

At 410, method 400 can include providing, by the one or more computing devices, the prediction data descriptive of the one or more predicted olfactory properties of the selected molecule as an output.

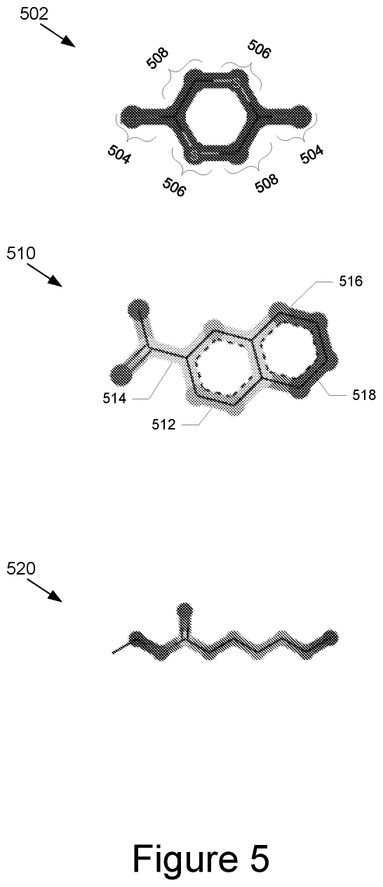

FIG. 5 depicts example illustrations for visualizing structural contribution associated with predicted olfactory properties according to example embodiments of the present disclosure. AS illustrated in FIG. 5, in some embodiments, the systems and methods of the present disclosure can provide output data to facilitate interpreting and/or visualizing which aspects of a molecule's structure most contributes to its predicted odor quality. For example, in some embodiments, a heat map could be generated to overlay the molecule structure, such as visualizations 502, 510, and 520, that provides indications of which portions of a molecule's structure are most important to the perceptual properties of the molecule and/or which portions of a molecule's structure are less important to the perceptual properties of the molecule. As an example, a heat map visualization, such as visualization 502, may provide indications that atoms/bonds 504 may be be most important to the predicted perceptual properties, that atoms/bonds 506 may be moderately important to the predicted perceptual properties, and that atoms/bonds 508 may be less important to the predicted perceptual properties. In another example, visualization 510 may provide indications that atoms/bonds 512 may be most important to the predicted perceptual properties, that atoms/bonds 514 may be moderately important to the predicted perceptual properties, and that atoms/bonds 516 and atoms/bonds 518 may be less important to the predicted perceptual properties. In some implementations, data indicative of how changes to a molecule structure would impact olfactory perception can be used to generate visualizations of how the structure contributes to a predicted olfactory quality. For example, iterative changes to the molecule's structure (e.g., a knock-down technique, etc.) and their corresponding outcomes can be used to evaluate which portions of the chemical structure are most contributory to the olfactory perception.

### Example Learned Graph Neural Network Embeddings

Some example neural network architectures described herein can be configured to build representations of input data at their intermediate layers. The success of deep neural networks in prediction tasks relies on the quality of their learned representations, often referred to as embeddings. The structure of a learned embedding can even lead to insights on the task or problem area, and the embedding can even be an object of study itself.

Some example computing systems can save the activations of the penultimate fully connected layer as a fixed-dimension “odor embedding”. The GNN model can transform a molecule's graph structure into a fixed-length representation that is useful for classification. A learned GNN embedding on an odor prediction task may include a semantically meaningful and useful organization of odorant molecules.

An odor embedding representation that reflects common-sense relationships between odors should show structure both globally and locally. Specifically, for global structure, odors that are perceptually similar should be nearby in an embedding. For local structure, individual molecules that have similar odor percepts should cluster together and thus be nearby in the embedding.

Example embedding representations of each data point can be produced from the penultimate-layer output of an example trained GNN model. For example, each molecule can be mapped to a 63-dimensional vector. Qualitatively, to visualize this space in 2D, principal component analysis (PCA) can optionally be used to reduce its dimensionality. The distribution of all molecules sharing a similar label can be highlighted using kernel density estimation (KDE).

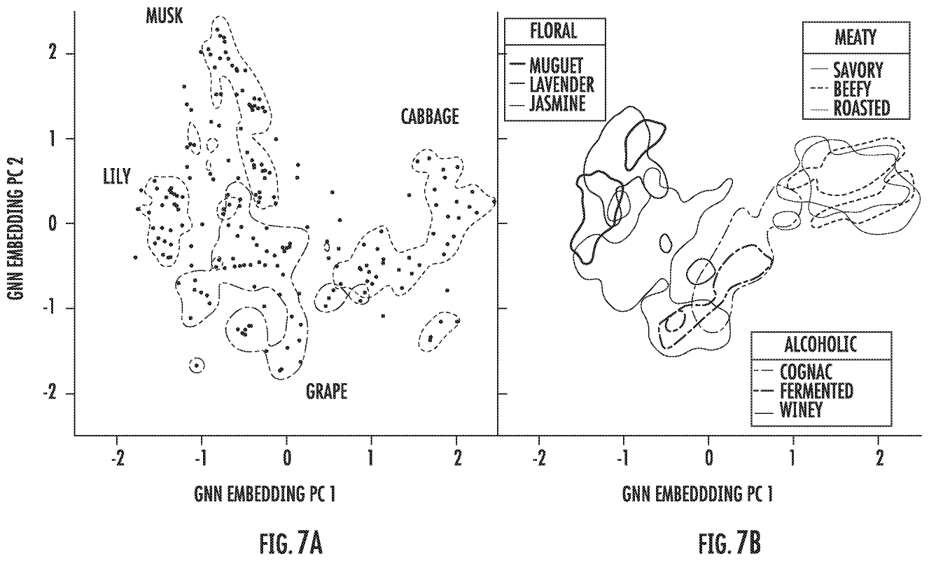

One example global structure of the embedding space is illustrated in FIG. 7. In this example, we find that individual odor descriptors (e.g. musk, cabbage, lily and grape) tend to cluster in their own specific region. For odor descriptors that co-occur frequently, we find that the embedding space captures a hierarchical structure that is implicit in the odor descriptors. The clusters for odor labels jasmine, lavender and muguet are found inside the cluster for the broader odor label floral.

FIG. 7 illustrates a 2D representation of a GNN model embeddings as a learned odor space. Molecules are represented as individual points. Shaded and contoured areas are kernel density estimates of the distribution of labeled data. A. Four odor descriptors with low co-occurrence have low overlap in the embedding space. B. Three general odor descriptors (floral, meaty, alcoholic) each largely subsume more specific labels within their boundaries. Example experiments have indicated that the generated embeddings can be used to retrieve molecules that are perceptually similar to a source molecule (e.g., using a nearest neighbor search over the embeddings).

### Example Transfer Learning

An odor descriptor may be newly invented or refined (e.g., molecules with the pear descriptor might be later attributed a more specific pear skin, pear stem, pear flesh, pear core descriptor). A useful odor embedding would be able to perform transfer learning to this new descriptor, using only limited data. To approximate this scenario, example experiments ablated one odor descriptor at a time from a dataset. Using the embeddings trained from (N−1) odor descriptors as a featurization, a random forest was trained to predict the previously held-out odor descriptor. We used cFP and Mordred features as a baseline for comparison. GNN embeddings significantly outperform Morgan fingerprints and Mordred features on this task, but as expected, still perform slightly worse than a GNN trained on the target odor. This indicates that GNN-based embeddings may generalize to predict new, but related, odors.

In another example, the proposed QSOR modeling approach can generalize to adjacent perceptual tasks, and capture meaningful and useful structure about human olfactory perception, even when measured in different contexts, with different methodologies

### ADDITIONAL DISCLOSURE

The technology discussed herein makes reference to servers, databases, software applications, and other computer-based systems, as well as actions taken and information sent to and from such systems. The inherent flexibility of computer-based systems allows for a great variety of possible configurations, combinations, and divisions of tasks and functionality between and among components. For instance, processes discussed herein can be implemented using a single device or component or multiple devices or components working in combination. Databases and applications can be implemented on a single system or distributed across multiple systems. Distributed components can operate sequentially or in parallel.

While the present subject matter has been described in detail with respect to various specific example embodiments thereof, each example is provided by way of explanation, not limitation of the disclosure. Those skilled in the art, upon attaining an understanding of the foregoing, can readily produce alterations to, variations of, and equivalents to such embodiments. Accordingly, the subject disclosure does not preclude inclusion of such modifications, variations and/or additions to the present subject matter as would be readily apparent to one of ordinary skill in the art. For instance, features illustrated or described as part of one embodiment can be used with another embodiment to yield a still further embodiment. Thus, it is intended that the present disclosure cover such alterations, variations, and equivalents.

1. A computer-implemented method, the method comprising:
obtaining, by one or more computing devices, a machine-learned graph neural network trained to predict olfactory properties of molecules based at least in part on chemical structure data associated with the molecules;
obtaining, by the one or more computing devices, a graph that graphically describes a chemical structure of a selected molecule;
providing, by the one or more computing devices, the graph that graphically describes the chemical structure of the selected molecule as input to the machine-learned graph neural network;
receiving, by the one or more computing devices, prediction data descriptive of one or more predicted olfactory properties of the selected molecule as an output of the machine-learned graph neural network; and
providing, by the one or more computing devices, the prediction data descriptive of the one or more predicted olfactory properties of the selected molecule as an output.

2. The computer-implemented method of claim 1, wherein obtaining, by the one or more computing devices, the machine-learned graph neural network comprises:
obtaining, by the one or more computing devices, training data comprising a plurality of example chemical structures, each example chemical structure labeled with one or more olfactory property labels that describe olfactory properties of the example chemical structure; and
training, by the one or more computing devices, the machine-learned graph neural network to predict olfactory properties of molecules based in part on the obtained training data.

3. The computer-implemented method of claim 1, further comprising:
generating, by the one or more computing devices, visualization data descriptive of a relative importance of one or more structural units of chemical structure of the selected molecule to the predicted olfactory properties associated with the selected molecule; and
providing, by the one or more computing devices, the visualization data in association with the prediction data indicative of the one or more olfactory properties.

4. The computer-implemented method of claim 1, further comprising:
generating, by the one or more computing devices, data indicative of how a structural change to the chemical structure of the selected molecule affects the predicted olfactory properties associated with the selected molecule.

5. The computer-implemented method of claim 1, wherein the prediction data indicative of the one or more olfactory properties of the selected molecule comprises an intensity of a particular olfactory property.

6. The computer-implemented method of claim 1, further comprising:
obtaining, by the one or more computing devices, a second graph that graphically describes a second chemical structure of a second selected molecule;
providing, by the one or more computing devices, the second graph that graphically describes the second chemical structure of the second selected molecule as input to the machine-learned graph neural network;
receiving, by the one or more computing devices, second prediction data descriptive of one or more second olfactory properties associated with the second selected molecule as an output of the machine-learned graph neural network; and
determining, by the one or more computing devices, one or more olfactory differences between the selected molecule and the second selected molecule based on a comparison of the prediction data for the selected molecule with the second prediction data for the second selected molecule.

7. The computer-implemented method of claim 1, further comprising determining, by the one or more computing devices through input of the graph that graphically describes the chemical structure of the selected molecule into the machine-learned graph neural network or an additional machine-learned graph neural network, data indicative of one or more of:
optical properties of the selected molecule;
gustatory properties of the selected molecule;
biodegradability of the selected molecule;
stability of the selected molecule; or
toxicity of the selected molecule.

8. The computer-implemented method of claim 1, wherein the graph that graphically describes the chemical structure of the selected molecule comprises a two-dimensional graph structure indicative of a two-dimensional representation of the chemical structure of the selected molecule.

9. The computer-implemented method of claim 1, wherein the graph that graphically describes the chemical structure of the selected molecule comprises a three-dimensional graph structure indicative of a three-dimensional representation of the chemical structure of the selected molecule, and wherein the method further comprises performing, by the one or more computing devices, one or more quantum chemical calculations to identify the three-dimensional representation of the chemical structure of the selected molecule.

10. The computer-implemented method of claim 1, further comprising:
performing, by the one or more computing devices, an iterative search process to identify an additional molecule that exhibits one or more desired olfactory properties, wherein the iterative search process comprises, for each of a plurality of iterations:
generating, by the one or more computing devices, a candidate molecule graph that graphically describes a candidate chemical structure of a candidate molecule;
providing, by the one or more computing devices, the candidate molecule graph that graphically describes the candidate chemical structure of the candidate molecule as input to the machine-learned graph neural network;
receiving, by the one or more computing devices, prediction data descriptive of one or more predicted olfactory properties of the candidate molecule as an output of the machine-learned graph neural network; and
comparing, by the one or more computing devices, the one or more predicted olfactory properties of the candidate molecule to the one or more desired olfactory properties.

11. The computer-implemented method of claim 1:
wherein the prediction data indicative of the one or more predicted olfactory properties of the selected molecule comprises a numerical embedding; and
the method further comprises identifying, by the one or more computing devices, other molecules that have olfactory properties that are similar to the predicted olfactory properties of the selected molecule by comparing the numerical embedding with other numerical embeddings output for the other molecules by the machine-learned graph neural network.

12. A computing device, comprising:
one or more processors; and
one or more non-transitory computer-readable media that store instructions that, when executed by the one or more processors, cause the computing device to perform operations, the operations comprising:
obtaining a machine-learned graph neural network trained to predict one or more olfactory properties of a molecule based at least in part on chemical structure data associated with the molecule;
obtaining graph data representative of a chemical structure of a selected molecule;
providing the graph data representative of the chemical structure as input to the machine-learned graph neural network;
receiving prediction data descriptive of one or more olfactory properties associated with the selected molecule as an output of the machine-learned graph neural network; and
providing the prediction data descriptive of the one or more predicted olfactory properties of the selected molecule as an output.

13. The computing device of claim 12, wherein obtaining the machine-learned graph neural network trained to predict one or more olfactory properties of a molecule further comprises:
obtaining training data comprising a plurality of example chemical structures, each example chemical structure labeled with one or more olfactory property labels that describe olfactory properties of the example chemical structure; and
training the machine-learned graph neural network to predict olfactory properties based in part on the obtained training data.

14. The computing device of claim 12 the operations further comprising:
generating data indicative how a structural change to the chemical structure of the selected molecule affects the predicted olfactory properties associated with the selected molecule.

15. The computing device of claim 12, the operations further comprising:
generating visualization data descriptive of a relative importance of one or more structural units of the selected molecule to the predicted olfactory properties associated with the selected molecule; and
providing the visualization data in association with the prediction data descriptive of one or more olfactory properties.

16. The computing device of claim 12, wherein the prediction data indicative of the one or more olfactory properties of the selected molecule comprises an intensity of a particular olfactory property.

17. The computing device of claim 12, the operations further comprising:
obtaining graph data representative of a chemical structure of a second selected molecule;
providing the graph data representative of the chemical structure of the second selected molecule as input to the machine-learned graph neural network;
receiving prediction data descriptive of one or more olfactory properties associated with the second selected molecule as an output of the machine-learned prediction model; and
determining one or more perceptual differences between the selected molecule and the second selected molecule.

18. The computing device of claim 12, the operations further comprising determining, based at least in part on graph data representative of the chemical structure, data indicative of one or more of:
optical properties of the selected molecule;
gustatory properties of the selected molecule;
biodegradability of the selected molecule;
stability of the selected molecule; or
toxicity of the selected molecule.

19. The computing device of claim 12, wherein the graph data representative of the chemical structure of the selected molecule comprises a graph structure indicative of a two-dimensional structure of the selected molecule.

20. The computing device of claim 12, wherein the graph data representative of the chemical structure of the selected molecule comprises a three-dimensional graph structure indicative of a three-dimensional representation of the chemical structure of the selected molecule, wherein the operations further comprise performing one or more quantum chemical calculations to identify the three-dimensional representation of the chemical structure of the selected molecule.
